(19) United States
(12) Patent Application Publication
(10) Pub. No.: US 2026/0241067 A1
(43) Pub. Date: Aug. 20, 2026

(54) METHOD AND SYSTEM FOR IODINATED, ARTIFICIAL INTELLIGENCE WOUND DRESSING

(71) Applicant: Iothane Medical Inc., Palo Alto, CA (US)

(72) Inventors: Eric LeVeen, Menlo Park, CA (US); Craig N. Lambert, San Jose, CA (US)

(73) Assignee: Iothane Medical Inc., Palo Alto, CA (US)

(21) Appl. No.: 19/542,549

(22) Filed: Feb. 17, 2026

Related U.S. Application Data
(60) Provisional application No. 63/759,363, filed on Feb. 17, 2025.

(51) Int. Cl. A61L 26/00; A61F 13/00; G16H 20/10

(52) U.S. Cl. CPC A61L 26/0085; A61F 13/00055; A61F 13/00059; G16H 20/10; A61L 2300/404

(57) ABSTRACT

A wound dressing system includes a wound dressing including a substrate and an iodinated polyurethane foam. The wound dressing system also includes one or more sensors coupled to the substrate, electronics operatively connected to the one or more sensors, a power source, and an output indicator coupled to the electronics. In some embodiments, a method of operating the wound dressing system includes detecting, using the one or more sensors coupled to the substrate, a parameter value corresponding to a state of the iodinated polyurethane foam, generating an electronic signal, using electronics operatively connected to the one or more sensors, corresponding to the state of the iodinated polyurethane foam, and displaying an output on an output indicator coupled to the electronics.

### CROSS-REFERENCES TO RELATED APPLICATIONS

This application claims priority to U.S. Provisional Patent Application No. 63/759,363, filed on Feb. 17, 2025, entitled “METHOD AND SYSTEM FOR IODINATED, ARTIFICIAL INTELLIGENCE (AI) WOUND DRESSING,” the disclosure of which is hereby incorporated by reference in its entirety for all purposes.

U.S. Pat. No. 5,810,755 and U.S. patent application Ser. No. 18/814,178, filed Aug. 23, 2024 are hereby incorporated by reference for all purposes.

### BACKGROUND OF THE INVENTION

Iodine has been demonstrated to provide antiseptic properties against pathogens. As a result, iodine is commonly used as a skin cleanser and antiseptic in preoperative hand scrubs.

Some wound dressings used, for example, to treat severe burns or chronic wounds can incorporate an antimicrobial agent to reduce the risk of infection of the wound. For example, silver salts can be utilized in antimicrobial wound dressings.

Despite the progress made in the treatment of wounds, there is a need in the art for improved methods and systems related to wound dressings.

### SUMMARY OF THE INVENTION

The present invention relates generally to wound dressing systems and methods, and in particular, to an iodinated, intelligent, wound dressing system and method. While the wound dressing protects wounds, including those resulting from burns and/or infection on the outside, it is occlusive to prevent loss of CO₂ from the surface of the wound. The occlusive property prevents the loss of carbon dioxide from the wound surface to maintain the acid pH of the wound. Oxygen is not released from oxyhemoglobin in an alkaline pH. Thus, by being occlusive, the film on the back of the dressing adds to the other embodiments to create the optimal environment for wound healing. The wound dressing, which may be hydrophilic, is capable of releasing therapeutic amounts of free iodine to the wound over prolonged periods in accordance with various embodiments of the invention.

In certain examples, the wound dressing may be replenished when medicants contained therein fall below a threshold and, in certain instances, may be replenished without removal from contact with the wound or during shortened period of time during which the dressing is moved. In short, some embodiments of the invention provide a wound dressing comprised of a hydrophilic, but insoluble material (for example, a polymer) and a compound capable of being reversibly complexed, with elemental iodine, thereby permitting the release of therapeutic amounts of free iodine into a wound that is brought into contact with the dressing. Additionally, embodiments of the present invention utilize the occlusive nature of the wound dressing to provide benefits not available using conventional techniques. One skilled in the art will understand that other types of material that have similar characteristics to those described above may also be used in other embodiments of the invention.

Numerous benefits are achieved by way of the present disclosure over conventional techniques. For example, embodiments of the present invention provide iodinated, intelligent, wound dressings that enable integrated sensing, analytics, and therapeutic control. The wound dressings described herein can utilize multi-modal sensing and analytics to provide closed-loop therapeutic delivery. The wound dressings are suitable for extended wear and use under compression. These and other embodiments of the disclosure, along with many of its advantages and features, are described in more detail in conjunction with the text below and corresponding figures.

### BRIEF DESCRIPTION OF THE DRAWINGS

FIG. 1 is a simplified schematic diagram illustrating a wound dressing with integrated electronics according to an embodiment of the present invention.

FIG. 2A is a simplified circuit diagram illustrating a system for measuring resistance for a wound dressing according to an embodiment of the present invention.

FIG. 2B is a simplified circuit diagram illustrating a resistance comparison circuit for a wound dressing according to an embodiment of the present invention.

FIG. 3 is a simplified circuit diagram illustrating a system for measuring resistance for a wound dressing in comparison to multiple reference values according to an embodiment of the present invention.

FIG. 4 is a simplified circuit diagram illustrating a multi-site resistance comparison circuit for a wound dressing including multiple reference values according to an embodiment of the present invention.

FIG. 5 is a simplified circuit diagram illustrating temperature measurement for a wound dressing according to an embodiment of the present invention.

FIG. 6 is a simplified circuit diagram illustrating pH measurement for a wound dressing according to an embodiment of the present invention.

FIG. 7 is a simplified schematic diagram illustrating inductive powering of electronics in a wound dressing according to an embodiment of the present invention.

FIG. 8A is a simplified cross-sectional schematic diagram illustrating an iodinated wound dressing according to an embodiment of the present invention.

FIG. 8B is a simplified cross-sectional schematic diagram illustrating a liquid port of the iodinated wound dressing illustrated in FIG. 8A according to an embodiment of the present invention.

FIG. 9 is a simplified schematic diagram illustrating integration of electronics into the wound dressing according to an embodiment of the present invention.

FIG. 10 is a simplified schematic diagram illustrating a top mounting of electronics for the wound dressing according to an embodiment of the present invention.

FIG. 11 is a simplified schematic diagram illustrating a wound dressing system including a transmitter/receiver and wound dressing according to an embodiment of the present invention.

FIG. 12 is a simplified schematic diagram illustrating a nested container system according to an embodiment of the present invention.

FIG. 13 is a simplified flowchart illustrating a method of operating a wound dressing system according to an embodiment of the present invention.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION OF SPECIFIC EMBODIMENTS

The incidence of infection in wounds may depend upon the causative factors in the type and extent of damage to the skin and tissue wounds resulting from accidental causes and the presence of foreign bodies. Examples include wounds exposed to environmental contaminants outdoors, when a foreign body is present or when the blood supply is compromised. When wounds are denuded of overlying skin, there is an additional tendency towards infection from airborne contamination. When a significant amount of underlying tissue is exposed, a risk of infection may exist, even in clean environments such as operating rooms. One skilled in the art will understand that that the incidence of infection even in clean operating rooms may be proportional to the length of time the tissue is exposed to air in many instances. Antibiotics may need a blood supply since they are dependent on this to reach devitalized tissue to stop infection.

In certain instances, the healing of tissue in this state may have prolonged exposure to air. Drying of wounded tissue during long periods of exposure to air devitalizes it, lowering resistance of the contaminated tissue to infection. Another factor detrimental to the healing of open wounds may be the loss of carbon dioxide to the air from the wound surface. Such loss may produce a respiratory alkalosis of the local tissue leading to an alkaline shift in the pH. This alkaline shift in pH may result in an undesirable shift in the hemoglobin/oxyhemoglobin dissociation curve which, in turn, stabilizes the oxyhemoglobin and inhibits its conversion to reduced hemoglobin at reduced oxygen tensions. One skilled in the art will understand that wounds are desirably maintained to a relatively acid pH so that the oxygen tension is raised. In many examples, applying appropriate topical treatments may benefit the healing of a wound.

Various products have been developed, intended to discourage infection when topically applied. Antibiotics and preparations containing silver salts or sulfonamide compounds have been used with some success. However, some wounds that result from burns often become infected notwithstanding such preventative application. Various prior art wound dressings have been developed to address the need to prevent wound infection, but have not heretofore demonstrated the required effectiveness. Iodine has been used advantageously to treat wounds and prevent infection by virtue of its germicidal, algaecide, sporicidal, amoebicidal, fungicidal, virucidal and bactericidal properties. Its properties are most effective, however, in the treatment of a wound or burn when iodine contacts with the surface uninterrupted. In some cases, continuous and repeated application of the iodine are utilized.

It has been shown clinically that a flush of aqueous free iodine with only 2 ppm may be sufficient to prevent peritonitis in patients receiving peritoneal dialysis as a treatment for renal failure. An iodine saline flush has also proved curative in other cases of peritonitis not related to dialysis. Furthermore, an aqueous iodine solution of 2 ppm iodine is non-irritating, even when placed directly in the eye. A 1 ppm iodine solution however is insufficient to kill organisms and is the meager amount released from 10% PVP Polyvinylpyrrolidone.

An iodine solution may be preferential and is the reason Betadine was taken off the market when it was reported that methicillin resistant Staphylococcus and Pseudomonas were growing in the solutions used to prep skin at surgery. One skilled in the art will understand that the saline will wash away glucose, which would otherwise tend to convert free iodine to inactive iodine. Subsequent studies have indicated that in certain instances, rather than the two parts per million shown effective in saline solution, a higher concentration between 5 ppm and 10 ppm of free iodine may be used when glucose and protein are not flushed away. In such a concentration, the iodine has been found to be bacterial, virucidal, fungicidal, and non-inflammatory, and non-irritating to tissue. Use of a lightly crosslinked polyurethane, which has been complexed with free iodine may be used to cause iodine to be released in therapeutic concentrations when exposed to an aqueous environment.

An open cell, polyurethane sponge can be effective in treating and preventing vaginitis when complexed with iodine and used as a tampon on the mucosa, and is well known for its effectiveness in preventing infection on undamaged external tissue, but the prior art does not teach nor advise the use of such an iodophor in direct contact with an open wound or burn. In various embodiments of the invention, a wound dressing is described that can protect an open wound or burned tissue from direct exposure to air, and can maintain the necessary aqueous environment and tissue needs for hydration but also can release medicants, such as iodine into the wounded tissue with which it is in contact. Iodine, in some embodiments is configured and used as a surgical drape, that in addition to providing a barrier against direct exposure to air, such a dressing contains iodine in its adhesive, rendering it bactericidal to the skin. This prevents infection of the wound from airborne contaminants in the operating room and the battlefield but also the commensal organisms populating our skin.

Accordingly, embodiments of the present invention provide a system and method for iodinated, intelligent, wound dressing with the advantages and features described herein. In particular, a smart, modular wound dressing system with integrated sensing, analytics, and therapeutic control is provided by embodiments of the present invention. More particularly, some embodiments provide medical wound dressings, and more particularly, smart wound dressing systems incorporating multi-modal sensing, analytics, and optional closed-loop therapeutic delivery, suitable for extended wear and use under compression.

In certain embodiments, a medical dressing system is described that includes a flexible polymeric substrate configured to contact tissue to form a wound dressing. The substrate may comprise a polyurethane foam, optionally iodinated, and may be covered by a polymeric film. Embedded within, attached to, or otherwise associated with the substrate can be a sensor array configured to monitor one or more wound environment parameters over time. The system is designed for continuous monitoring over extended durations, including periods of at least 5-7 days.

The wound-contact layer can include a flexible polyurethane structure configured to contact tissue. In certain embodiments, the polyurethane includes open-cell or reticulated foam. The wound-contact layer may incorporate antimicrobial agents, iodinated compounds, hydrophilic or anti-adhesion components, and the like. Multilayer constructions are included within the scope of the present invention since, in some embodiments, the dressing comprises multiple layers including: a wound-contact layer, a sensor layer, a signal-routing layer, a protective outer film, or the like. Conductive pathways may be embedded within the polymeric structure or formed using flexible circuitry.

The sensor array may include one or more sensors configured to monitor wound and peri-wound conditions, including but not limited to: Temperature, Moisture and Hydration, pH, Bioimpedance, Oxygenation, Carbon Dioxide (CO₂), Exudate Volume and Flow, Biomarker Detection, or the like.

Temperature: In relation to temperature, two or more temperature sensors may be included, at least one sensor can be positioned near the wound center, at least one sensor can be positioned near the wound edge or peri-wound skin, differential temperature analysis can be used to detect inflammation, infection, or ischemia, and the like. Temperature sensors may include thin-film thermistors or silicon temperature ICs on flexible substrates.

Moisture and Hydration: In relation to moisture and hydration, moisture levels may be measured using capacitive humidity electrodes or impedance-based sensors to assess hydration and maceration risk.

pH: In relation to pH, pH measurement may be performed using optical pH sensing patches, potentiometric electrodes, hybrid optical-electrical configurations, or the like. In certain embodiments, redundant pH sensing is used to compensate for optical drift.

Bioimpedance: In relation to bioimpedance, electrical impedance spectroscopy may be used to assess tissue edema, monitor inflammation, evaluate dressing integrity, or the like. Multiple electrodes (e.g., 2-4 electrodes) may operate at different frequencies.

Oxygenation: In relation to oxygenation, tissue oxygenation may be assessed using reflective photoplethysmography (PPG), near-infrared spectroscopy (including NIRS-like techniques), or the like. These measurements may be used to evaluate perfusion and ischemic conditions.

Carbon Dioxide (CO₂): In relation to CO₂, in some embodiments, CO₂ concentration is monitored as a metabolic indicator. CO₂ sensing may be performed using optical sensors positioned behind gas-permeable, liquid-impermeable membranes, or the like.

Exudate Volume and Flow: In relation to exudate volume and flow, exudate flow and volume may be measured using microfluidic channels, capacitive fill sensors, impedance-based measurements, or the like.

Biomarker Detection: In relation to biomarker detection, the system may include chemical, enzymatic, or fluorescence-based sensors to detect biomarkers associated with infection, inflammation, or healing. In certain embodiments, a disposable chemical sensing patch is paired with a reusable optical reader.

### Modular Disposable/Reusable Architecture

In some embodiments, the system is configured such that the wound-contact dressing is disposable or one or more sensors or electronic modules are reusable. Reusable modules may include optical emitters and detectors, signal processing electronics, power sources, wireless transmitters, or the like. Electrical or mechanical coupling may be achieved using magnetic interfaces, pogo-pin connectors, conductive adhesive interfaces, capacitive or inductive coupling, or the like. Power sources, including lithium batteries, can thus be isolated from the wound environment.

### Edge Seal Integrity Monitoring

The system can recognize the edge seal between dressing and surrounding skin as a critical functional zone. In certain embodiments, sensors are positioned at or near the dressing edge to monitor moisture accumulation, adhesive lift-off, impedance discontinuities, protein accumulation, early bacterial ingress, or the like at the periphery of the wound dressing. Edge seal integrity monitoring may be used to predict maceration, detect seal failure, trigger alerts, indicate dressing replacement, or the like.

### Operation Under Compression

The system is configured to remain operational under compression bandages, beneath negative pressure wound therapy interfaces, during patient movement, or the like. Sensors and conductive elements can be mechanically isolated or elastically coupled to maintain function under compressive loads.

### Closed-Loop Therapeutic Delivery

In certain embodiments, the system includes a therapeutic delivery component. Therapeutic agents may include antimicrobial agents, iodinated compounds, nitric oxide or nitric oxide donors, fluids for hydration or irrigation, growth-modulating or immunomodulating compounds, or the like. Therapeutic delivery may be triggered based on sensor-derived thresholds and may be controlled via time-based release, electrically triggered release, thermally triggered release, feedback-controlled algorithms, or the like.

### Thermal Therapy

In some embodiments, a heating element is integrated into the dressing to increase tissue temperature, improve perfusion, treat frostbite, enhance antimicrobial activity, or the like. Heating may be resistive and feedback-controlled using embedded temperature sensors. Thermal therapy can also include passing cool fluid through the wound to slow or prevent tissue destruction due to trapped heat.

### Data Processing and Analytics

Sensor data may be stored locally, transmitted wirelessly to an external device, processed in real time, or the like. Analysis may be rule-based, model-based, AI-driven, or the like. For example, AI-based analysis may include machine-learning models trained on multimodal wound data to detect early infection, predict healing trajectories, optimize dressing change timing, adjust therapeutic delivery, or the like.

### External Devices and Communication

The system may communicate with a handheld reader, a smartphone, a clinical workstation, a cloud-based analytics platform, or the like. External systems may provide visualization, alerts, and integration with electronic medical records.

### Clinical Use Cases

The system is configured for use in chronic wounds, surgical wounds, traumatic wounds, military wounds, burns, frostbite injuries, ischemic wounds, graft monitoring, or the like.

Thus, embodiments of the present invention provide a modular, intelligent wound dressing platform capable of continuous sensing, analytics, and optional closed-loop therapy, with particular emphasis on edge seal integrity, compression-safe operation, and extended wear duration.

### Embedded Electronics for an Intelligent Wound Dressing

An intelligent medical dressing can drastically improve outcomes of treatments for burns and many other types of wounds, especially in non-sterile environments (e.g., battlefields, outside urban and rural areas, unsanitary indoor settings). Such a dressing utilizes circuitry to monitor a range of parameters and alert the patient or caregiver to variations of any of those parameters outside predefined limits. Those parameters could include the concentration of infection-preventing medicine, for example iodine, the pH level, and the temperature of the wound, any of which could indicate the presence of infection. The results of the measurements can be presented in a variety of ways, including light-emitting diodes (LEDs) on the dressing, an audible alarm from a buzzer on the dressing, or a separate device, such as a cell phone or dedicated hand-held device that communicates with the dressing electronics through radio frequency identification (RFID) or a proprietary protocol.

An important aspect for the dressing electronics is power management because electronics rely on electrical power during operation. Options include a small battery on the dressing or circuitry in the dressing that harvests energy from a nearby source, such as a cell phone with an RFID system or a hand-held companion device. Depletion of the infection-suppressing substance in the dressing or advance of infection in the wound proceed sufficiently slowly that the parameters do not usually utilize continuous monitoring. Measurements can be made minutes of hours apart without risk to the patient, thus conserving battery life in the battery-powered option.

Some embodiments of the present invention provide an intelligent dressing that is infused with iodine or another bactericidal substance with embedded battery-powered circuitry that monitors the abundance, for example, of the iodine at one point at the wound-side of the dressing.

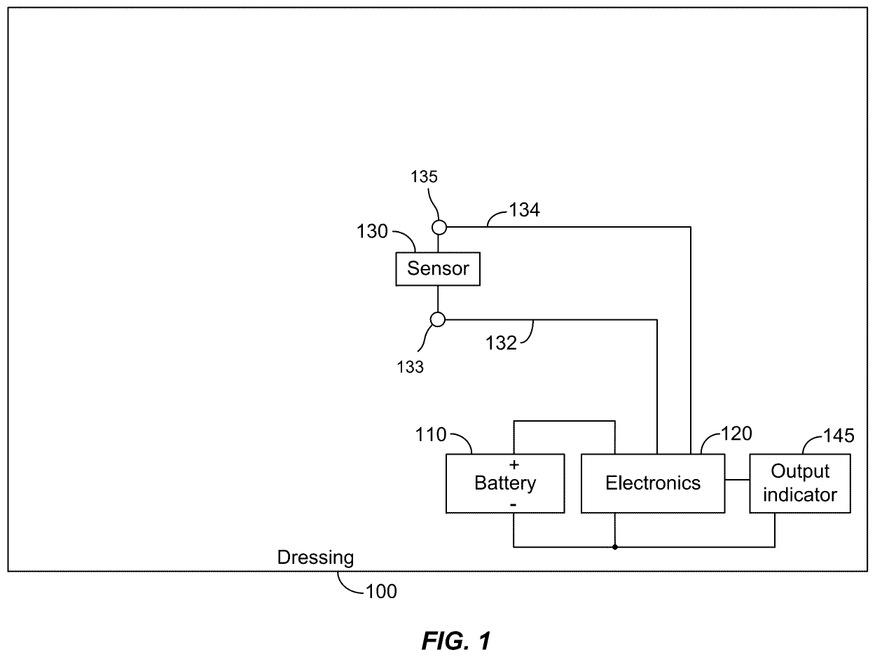

FIG. 1 is a simplified schematic diagram illustrating a wound dressing with integrated electronics according to an embodiment of the present invention. As illustrated in FIG. 1, wound dressing 100 includes sensor 130 that measures properties of the wound dressing at site 133 in comparison to the properties of the wound dressing at site 135 using current flowing through lead 132 and lead 134. As shown in FIG. 1, electronics 120 are powered by battery 110 and the output produced by electronics 120 is displayed using output indicator 145. Additional description related to the application of these various elements to particular measurements of the properties of wound dressing 100 and output of data related to these measurements is provided throughout the specification.

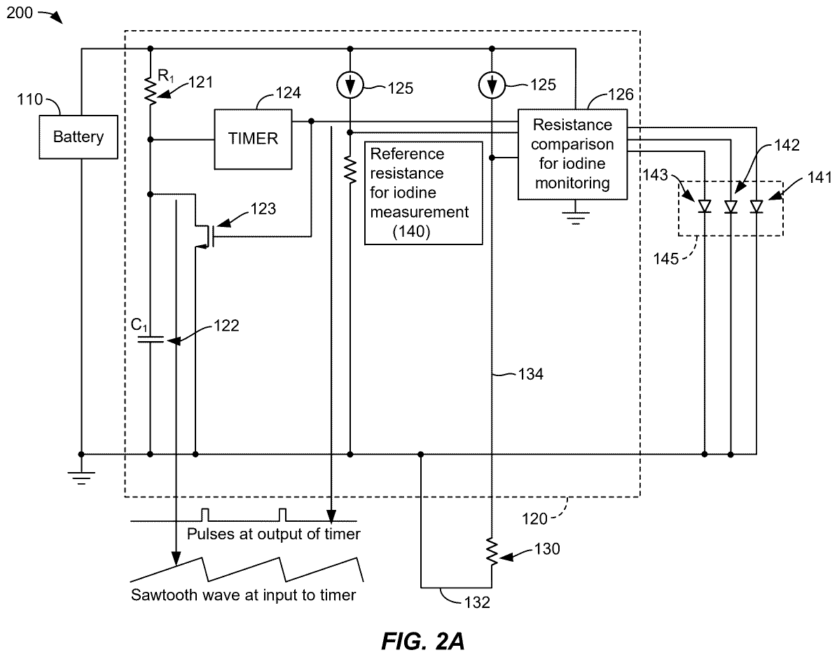

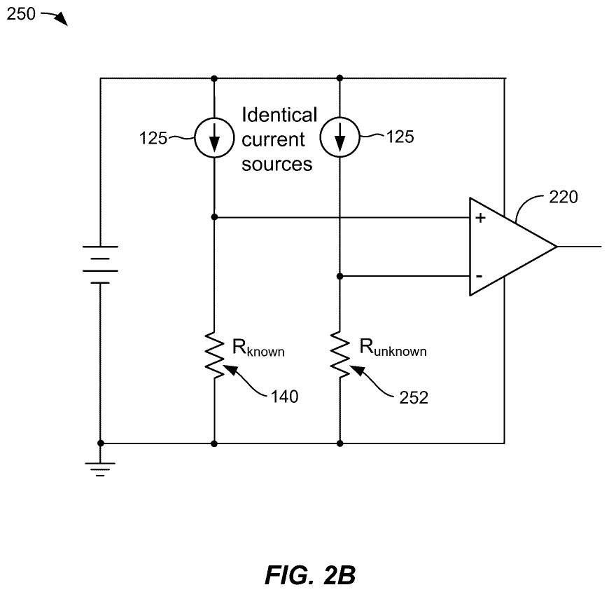

FIG. 2A is a simplified circuit diagram illustrating a system for measuring resistance for a wound dressing according to an embodiment of the present invention. As illustrated in FIG. 2A, simplified circuit diagram 200 can be used to illustrate resistance measurements for a wound dressing according to an embodiment of the present invention. In the proposed implementation shown in FIG. 2A, the frequency of the measurements is set by the time constant of resistor R1 121 and capacitor C1 122. This can be set by the manufacturer or made field-adjustable by replacing the fixed resistor R1 121 with a variable resistor. This field-adjustable feature can be useful in an environment in which the risk of infection is known to be high, resulting in shorter times between measurements, or low, resulting in longer times between measurements. Reducing the frequency of the measurements will also increase battery life.

A microswitch (not shown) can also be mounted near the electronic circuit for on-demand status checks. The state of the iodine in the dressing can be displayed periodically using timer 124 or at the closing of the microswitch by one of three LEDs: a green LED 141 indicates that the iodine level is acceptable, a yellow LED 142 indicates that the measured resistivity is too high (i.e., indicating that the iodine is depleted), and a red LED 143 indicates that the resistivity is too low (i.e., the wound dressing is contaminated by blood or other liquid from the wound). The last of these three options is explored in more detail later in this disclosure.

The simplest form of iodine measurement is that of the concentration-dependent resistivity of the solution corresponding to reference resistor 140 and output using output indicator 145. The reference against which the concentration-dependent resistivity of the solution is compared can be a reference resistor 140, which can be implemented using a physical resistor or, in some embodiments, a reservoir of the proper concentration of solution. In integrated circuits, with their poor component precision but precise proportionality, a comparison method such as shown in FIG. 2B is common to determine whether an unknown resistance is greater or less than a known resistance, also referred to as a reference resistance.

During operation, battery 110 is used to power the circuit and a sawtooth wave is produced at the input to timer 124. The output of timer 124 is a series of pulses as transistor 123 switches between off and on states. Thus, periodically, the resistance comparison for iodine monitoring using comparison circuit 126 can be performed in relation to the resistance of reference resistor 140 based on the resistance values resulting from current flow through each current source 125 and sensor 130 (e.g., a resistor). Components of electronics 120 can be integrated into the wound dressing in one of several manners as discussed more fully herein.

FIG. 2B is a simplified circuit diagram 250 illustrating a resistance comparison circuit for a wound dressing according to an embodiment of the present invention. As illustrated in FIG. 2B, with identical currents, i.e., current source 125, delivered to both reference resistor 140 and resistance 252, also referred to as a test resistance or an unknown resistance, a voltage comparator 220 can determine which resistance is higher and thereby determine whether the amount of remaining iodine in the dressing is above or below the required level. A voltage reading above a maximum level can indicate that the iodine solution needs to be replenished, because the resistivity of the solution will increase as the electrolyte is used up. A voltage measurement below the target resistivity of the solution can show that the dressing has been saturated by exudate from the wound or replenished with a higher concentration of iodine than intended. In either case, the wound dressing may need to be replaced.

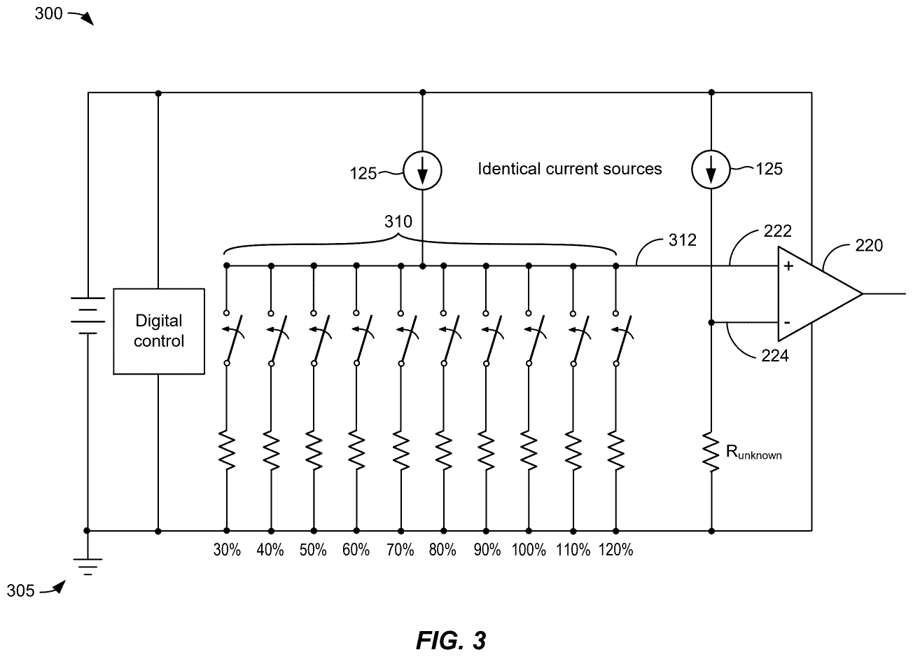

FIG. 3 is a simplified circuit diagram illustrating a system 300 for measuring resistance for a wound dressing in comparison to multiple reference values according to an embodiment of the present invention. In the implementation illustrated in FIG. 3, two electrodes can be placed at a location on the wound side of the wound dressing. A first electrode is connected to the signal ground reference 305. The other electrode is connected to input 224 of voltage comparator 220. Input 222 of voltage comparator 220 is connected to the output 312 of a multiplexer 310 that presents a sequence of monotonically increasing or decreasing voltages derived from an electrical analog of the intended iodine concentration. As an example, the sequence of voltages could correspond to thirty, forty, fifty, sixty, seventy, eighty, ninety, one hundred, one hundred ten, and one hundred twenty percent of the target iodine concentration. If the voltage comparator 220 determines that the wound-site concentration of iodine is above one hundred percent of the desired level, the red LED 143 illustrated in FIG. 1 is illuminated or blinks for a short time. If the concentration is below the minimum effective concentration, the yellow LED 142 illustrated in FIG. 1 is illuminated or blinks. Otherwise, the green LED 141 illustrated in FIG. 1 is illuminated or blinks.

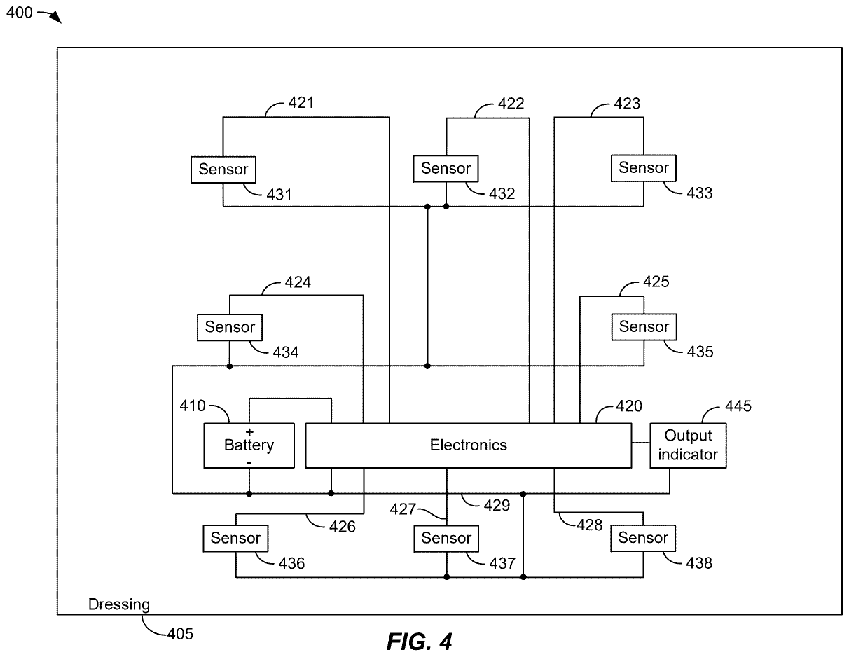

While the single wound site monitor would work for a small wound, it is possible that it may not provide enough information for a larger wound dressing. Accordingly, for larger wound dressings, embodiments of the present invention provide a wound dressing as discussed in relation to FIG. 4.

FIG. 4 is a simplified circuit diagram 400 illustrating a multi-site resistance comparison circuit for a wound dressing 405 according to an embodiment of the present invention. In this embodiment, electrodes embedded at multiple sites in the wound dressing connect to a multiplexer. Referring to FIG. 4, a first sensor 431 embedded in the wound dressing at a first site (e.g., characterized by resistance R1) is connected to electronics 420 using lead 421. Similarly, second sensor 432, third sensor 433, fourth sensor 434, fifth sensor 435, sixth sensor 436, seventh sensor 437, and eighth sensor 438 located at second, third, fourth, fifth, sixth, seventh, and eighth sites, respectively, are connected to electronics 420 using second lead 422, third lead 423, fourth lead 424, fifth lead 425, sixth lead 426, seventh lead 427, and eighth lead 428, respectively. Electronics 420 are powered by battery 410 connected to common ground 429 and output is produced on output indicator 445.

In this way, the operation of the circuit described in relation to FIG. 2A can be repeated several times, once for each site. The results can be displayed through LEDs at each site. Alternatively, the results can be averaged with analog or digital circuitry and the results of that operation can be displayed with a single set of three LEDs. In a particular embodiment, the LEDs corresponding to the highest and lowest concentrations would be lit or activated. If all measurements are in the acceptable range, only the green LED would be lit or activated. If any site reads a low resistance, the red LED is lit. Any site that measures a high resistance causes the yellow LED to light.

In some embodiments, the implementations illustrated in FIG. 3, i.e., a wound dressing including multiple reference values and the FIG. 4, i.e., a multi-site resistance comparison circuit for a wound dressing, can be combined to provide a wound dressing in which multiple sites are utilized, each of the different sites having multiple reference values. One of ordinary skill in the art would recognize many variations, modifications, and alternatives.

Instead of, or in addition to, the iodine concentration measurements, thermocouples can be implanted at the wound side of the wound dressing, in contact with the patient's skin to monitor for increases in temperature that might indicate the onset of infection. Because skin temperature varies with ambient temperature, exposure to sunlight or other heat sources, and other factors, a minimum of two thermocouples can be utilized to make a differential measurement: one or more at the wound site and another “reference” thermocouple near the edge of the wound dressing to sense the temperature of uninjured skin. An alternative approach, which may be cheaper and/or simpler to implement, would have the silicon die comprising the on-dressing electronics in contact with the patient's skin, permitting direct temperature measurement based on the temperature of the die. This solution does not provide the differential aspect of the multiple-thermocouple approach, but it might be appropriate in an environmentally-controlled setting. Alternatively, or in addition, pH sensors can be embedded at the wound side of the wound dressing to detect chemical changes in the wound.

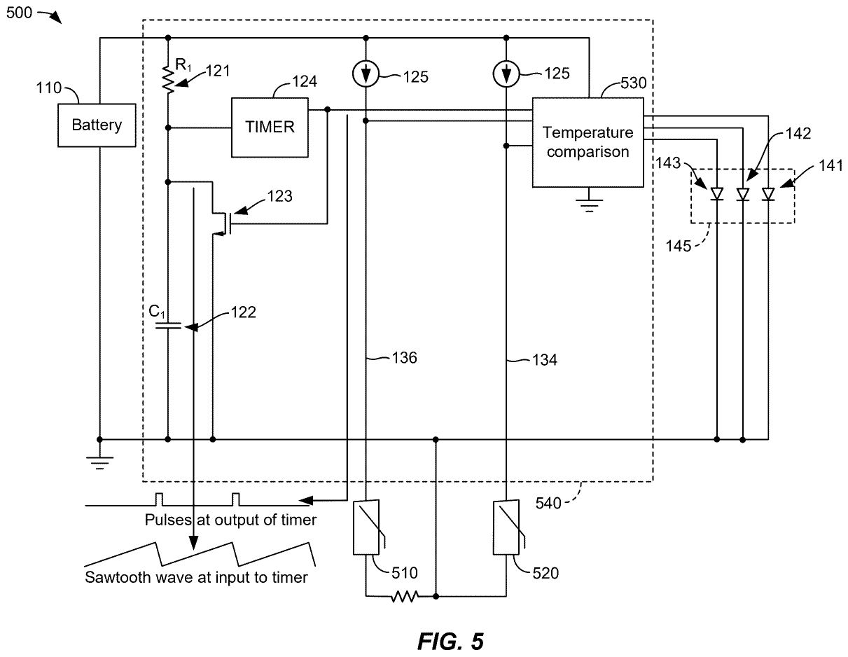

FIG. 5 is a simplified circuit diagram illustrating temperature measurement for a wound dressing according to an embodiment of the present invention. The circuit diagram 500 illustrated in FIG. 5 shares common elements with the circuit diagram 200 illustrated in FIG. 2A and the discussion provided in relation to the circuit diagram illustrated in FIG. 2A is applicable to the circuit diagram illustrated in FIG. 5 as appropriate. In FIG. 5, a temperature comparison 530 is implemented using thermistor 510 as the reference temperature and thermistor 520 as the measurement temperature. Thermistor 510 is connected to current source 125 by lead 136 and thermistor 520 is connected to current source 125 by lead 134. Thus, a temperature of a particular site can be monitored using the circuit illustrated in FIG. 5.

In other implementations, a multi-site temperature measurement can be made by modifying the electronics 540 of the circuit diagram 500 by incorporating multi-site measurements as illustrated in FIG. 4. Further, multi-site temperature measurements can be implemented using multiple thermistors at each of the sites by replacing the series of resistors illustrated in FIG. 3 with a series of thermistors and incorporating this multi-reference value concept into circuit diagram 500. One of ordinary skill in the art would recognize many variations, modifications, and alternatives.

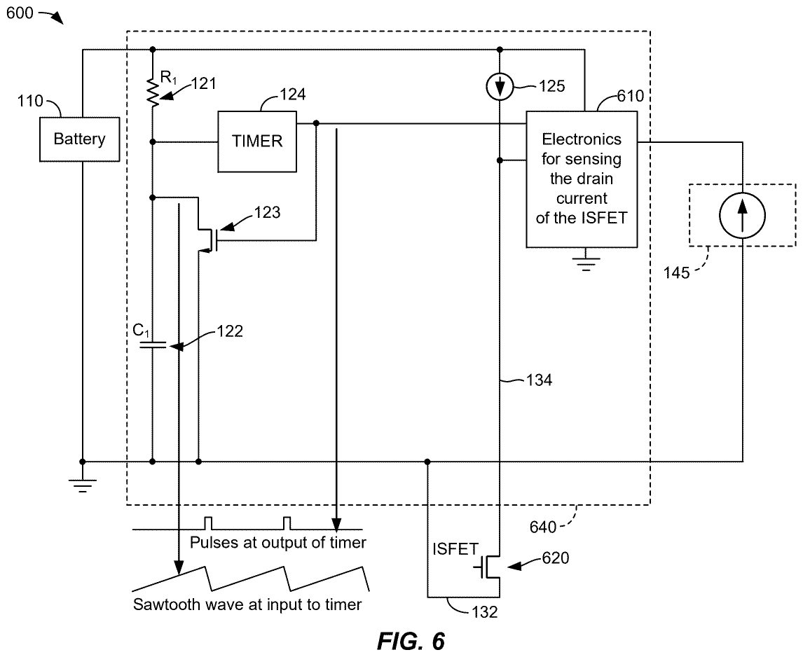

FIG. 6 is a simplified circuit diagram illustrating pH measurement for a wound dressing according to an embodiment of the present invention. The circuit diagram 600 illustrated in FIG. 6 shares common elements with the circuit diagram 200 illustrated in FIG. 2A and the discussion provided in relation to the circuit diagram illustrated in FIG. 2A is applicable to the circuit diagram illustrated in FIG. 6 as appropriate. In FIG. 6, a pH comparison is implemented using electronics 610 for sensing the drain current on ion-sensitive field-effect transistor (ISFET) 620. Thus, a pH of a particular site can be monitored using the circuit illustrated in FIG. 6.

In other implementations, a multi-site pH measurement can be made by modifying the electronics 640 of the circuit diagram 600 by incorporating multi-site measurements as illustrated in FIG. 4. Further, multi-site pH measurements can be implemented using multiple ISFETs at each of the sites by replacing the series of resistors illustrated in FIG. 3 with a series of ISFETs and incorporating this multi-reference value concept into circuit diagram 600. One of ordinary skill in the art would recognize many variations, modifications, and alternatives.

One advantage of the embodiments described herein is that the battery can power a real-time clock to monitor the elapsed time since the wound dressing was placed on the wound. This information would be valuable for caregivers. Some embodiments utilize a battery in each wound dressing, which increases the cost of the wound dressing and could amount to a number of batteries being disposed of annually, for example, in medical incinerators. In a field setting this may be unavoidable. However, the environmental impact associated with burning batteries can be eliminated by designing the circuity in the dressing to harvest energy from a companion device. In this implementation, the wound dressing would not require a timer to wake up the electronics for periodic monitoring or a switch for on-demand measurements. Rather, the companion device would contain an electrical coil that would be energized by closing a switch. When brought into proximity to the wound dressing, that coil and a similar coil in the wound dressing would form a transformer and electrical energy would be transmitted to the wound dressing, which would activate the electronics in the wound dressing. Further operation would proceed as described above until the switch on the transmitter turned off or the transmitter was moved away from the wound dressing. The transmitter would work with all wound dressings without requiring pairing or other authorization, so a small hospital or medical practice would need only one or a few.

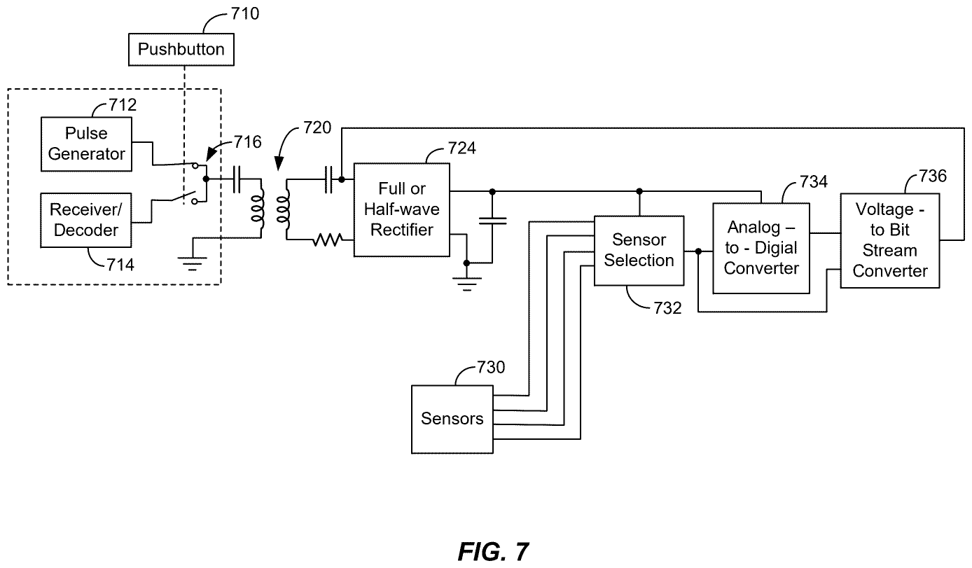

Building on this implementation, the indicator LEDs could be moved from the wound dressing to the transmitter. The transmitter would activate the electronics in the wound dressing, as above, and the measurements would proceed as previously described. In this implementation, however, the wound dressing electronics would also include a capacitor of sufficient capacity to maintain enough voltage across the wound dressing circuity such that when the transmitter is turned off, but left in proximity to the wound dressing, the wound dressing would transmit the results of the measurements to the transmitter. Circuitry on the transmitter would decode and display the results. This implementation further reduces the cost of the wound dressing, since a capacitor can be cheaper than a battery and a coil can be cheaper than three LEDs. An example is shown in FIG. 7.

FIG. 7 is a simplified schematic diagram illustrating inductive powering of electronics in a wound dressing according to an embodiment of the present invention. As illustrated in FIG. 7, a pulse generator 712 is used to generate pulses that are transmitted to transformer 720.

In order to initiate a measurement, push button 710 is operated to close switch 716, which connects the pulse generator 712 to the full-wave or half-wave rectifier 724 in order to power the electronics including the components discussed below.

On the dressing side of the inductive powering system, full-wave or half-wave rectifier 724 is utilized to communicate with sensors 730 that are operated by sensor selection block 732. Thus, the sensor(s) to be measured can be selected during operation. The sensor selection block 732 is also connected to analog-to-digital (A/D) converter 734 that can be used to sample and create a digital value corresponding to the measurements and voltage-to-bit stream converter 736 that is used to convert the digital value to a bit stream that can be transmitted to the receiver/decoder 714.

When the push button 710 is released, the receiver/decoder 714 is connected to transformer 720, which enables reading of the measurements made by the sensors in the dressing and communicated by the bit stream.

During operation, peak detectors can capture the highest and lowest measurements while the transmitter was powering the wound dressing electronics. After the power transmission from the pulse generator is turned off, a capacitor can maintain a sufficient voltage across the wound dressing electronics to permit continued operation. During this phase, the DC voltage output of first peak detector would be translated to a digital bit-stream, the duty cycle (i.e., the ratio of widths of high and low voltages) of which would depend on the DC voltage. After a set period of time, the input of the bit-stream generator would switch to the second peak detector. This process, switching between the first peak detector and the second peak detector and generating a bit-stream that alternates between two sequences of duty-cycles, would continue until the charge on the capacitor decayed below the minimum required to support operation (e.g., after several seconds).

Meanwhile, after the pulse generator stops sending power to the wound dressing electronics, it can become a receiver. It senses the bit-stream sent from the wound dressing and integrates the pulses to recreate the DC voltages, or analogs thereof, stored in the peak detectors in the wound dressing electronics. The two DC levels recreated in the transmitter (now functioning as a receiver) are captured by peak detectors in the transmitter and compared to voltage references. Those comparisons result in the illumination of LEDs on the transmitter that correspond to the minimum and maximum measurements in the wound dressing.

The monitoring approaches outlined above are not exhaustive, either in the types of tests, their implementations, or how the results are displayed or captured. In addition, rather than determining the iodine concentration by the resistivity of the liquid in the wound dressing, it could instead be sensed with an iodine-specific, ion-selective sensor.

In a hospital setting, other approaches are possible. One example uses a smartphone or dedicated electronic device to assign a unique identification code to each wound dressing after it is applied. The wound dressing could use Bluetooth or some other communication protocol to send periodic messages to a receiver in each patient's room or throughout the hospital. The messages are then transmitted to the appropriate nurses' station where they are monitored and electronically logged. When a fault condition is reported by the wound dressing (e.g., bactericidal solution too high or low, elevated temperature at the wound site, or pH outside the acceptable range, or the like) the responsible nurse is alerted and the wound dressing is evaluated for replacement. This reduces the amount of time nurses spend checking wound dressings.

An interesting property of blood is that it is an electrically conductive liquid (i.e., plasma) containing quanta of another conductive liquid encapsulated within non-conductive membranes (i.e., red blood cells). At DC and reasonably low frequencies, only the plasma is involved in the transmission of current through the blood, i.e., the conductance and reactance of the blood is constant with frequency. The red blood cells act as resistors electrically isolated from the conductive plasma by capacitors (i.e., the red blood cells, with conductive media on either side of an insulating dielectric). At sufficiently high frequencies, the electrical admittance of the red blood cells causes the red cells to contribute to the conduction of current through the blood, causing its overall AC conductivity (i.e., admittance) to increase. This is useful when monitoring the level of iodine solution in the wound dressing since the contribution to conductance due to the iodine can easily be separated from the contribution due to blood infiltration into the wound dressing. The measurements and mathematical adjustments can be made directly by the wound dressing electronics or in an app on a smartphone or other companion device.

A direct application of this measurement tool is that wound dressings can easily be identified as covering more-or less-heavily bleeding wounds, alerting medical caregivers to the need for the addition of clotting agents or other therapeutic measures.

Taking this a step further, the wound dressing(s) can also monitor relative changes in the admittance of the blood. With heavy bleeding, the level of both plasma and red cells decreases. The body compensates by pulling water from the body's tissues to maintain blood pressure, so overall the AC and DC conductances decrease. If no wound dressing reports heavy bleeding, this would indicate the presence of internal bleeding, likely requiring surgical intervention. This could also be useful in determining the need for blood products.

### Iodinated Dressing Specifications

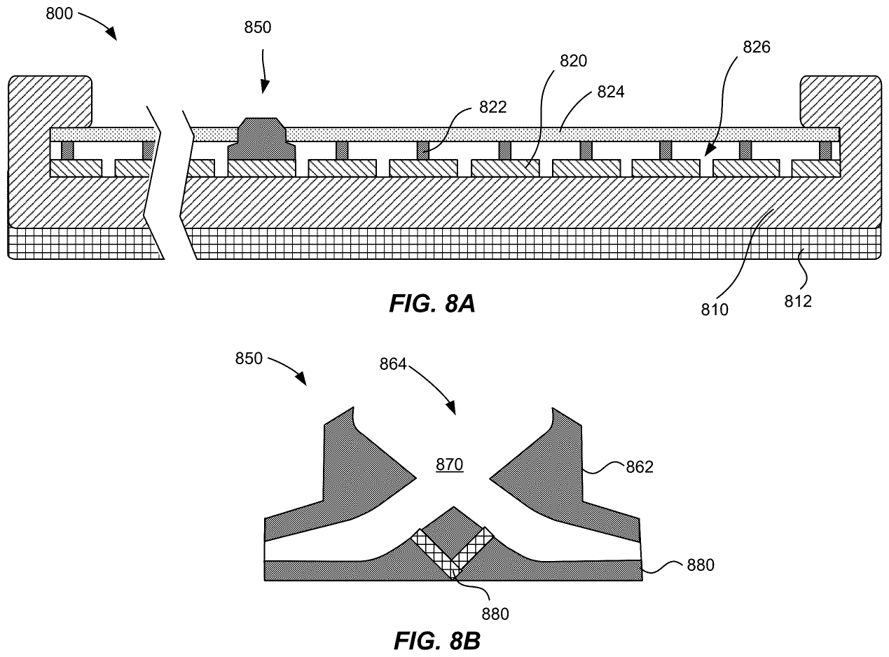

FIG. 8A is a simplified cross-sectional schematic diagram illustrating an iodinated wound dressing according to an embodiment of the present invention. FIG. 8B is a simplified cross-sectional schematic diagram illustrating a liquid port of the iodinated wound dressing illustrated in FIG. 8A according to an embodiment of the present invention.

Referring to FIG. 8A, the iodinated wound dressing 800, which can have an overall height of 9 mm, includes iodinated foam 810, also referred to as an iodinated sponge, having a total thickness on the order of 3 mm and a substrate 812 having a thickness on the order of 3 mm. The iodinated foam 810 supports a perforated inner polyurethane film 820 having a thickness on the order of 1 mm, a set of polyurethane columns 822, and an outer polyurethane film 824 separated from the perforated inner polyurethane film 820 by the set of polyurethane columns 822. The set of polyurethane columns 822 can have a height on the order of 1 mm, providing a vacuous inner chamber 826 for fluid infusion.

A liquid port 850 of the iodinated wound dressing 800 is illustrated in FIG. 8B and includes a base 860 coupled to a portion of perforated inner polyurethane film 820 illustrated in FIG. 8A and a silicone annulus 862 coupled to outer polyurethane film 824 illustrated in FIG. 8A. Fluid channel 870 is formed between base 860 and silicone annulus 862 to allow fluid (e.g., an iodine solution) to flow through input port 864 in silicone annulus 862 to vacuous inner chamber 826 illustrated in FIG. 8A. Stop 880 can be formed from a high strength material and utilized to prevent passing of a needle, e.g., a needle being used to recharge the wound dressing with an iodine solution, from passing through liquid port 850 and into the patient's skin.

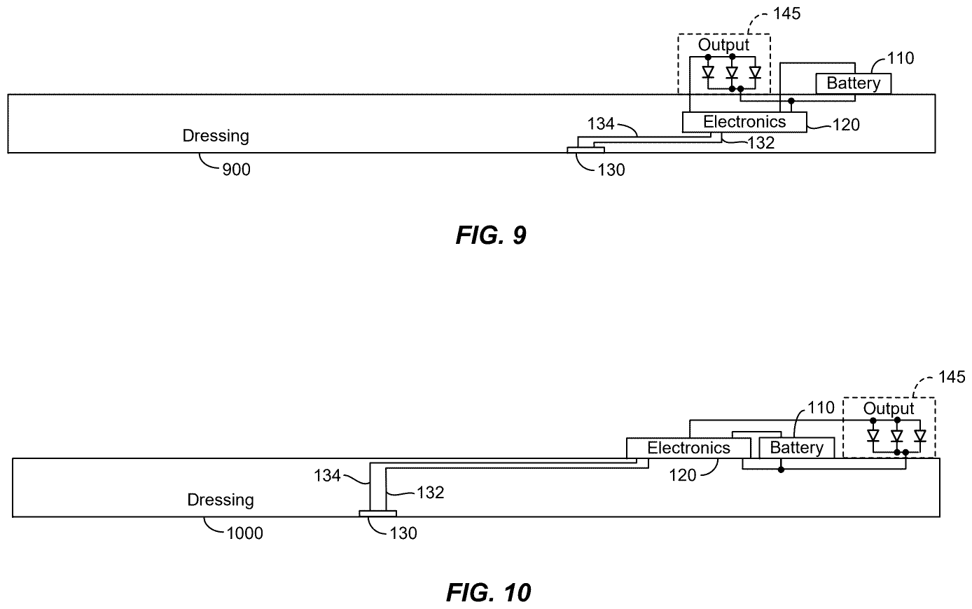

FIG. 9 is a simplified schematic diagram illustrating integration of electronics into the wound dressing according to an embodiment of the present invention. As illustrated in FIG. 9, dressing 900 can integrate electronics 120 inside the wound dressing with sensor 130 mounted on one surface of the wound dressing (e.g., the side of the wound dressing applied to the skin) and output indicator 145 and battery 110 mounted on the opposing side of the wound dressing (e.g., the side of the wound dressing facing away from the skin).

FIG. 10 is a simplified schematic diagram illustrating a top mounting of electronics for the wound dressing according to an embodiment of the present invention. In the embodiment illustrated in FIG. 10, dressing 1000 can utilize a top-mount configuration in which sensor 130 is mounted on one surface of the wound dressing (e.g., the side of the wound dressing applied to the skin) and, in addition to output indicator 145 and battery 110, which are mounted on the opposing side of the wound dressing (e.g., the side of the wound dressing facing away from the skin), electronics 120 can also be mounted on the opposing side of the wound dressing.

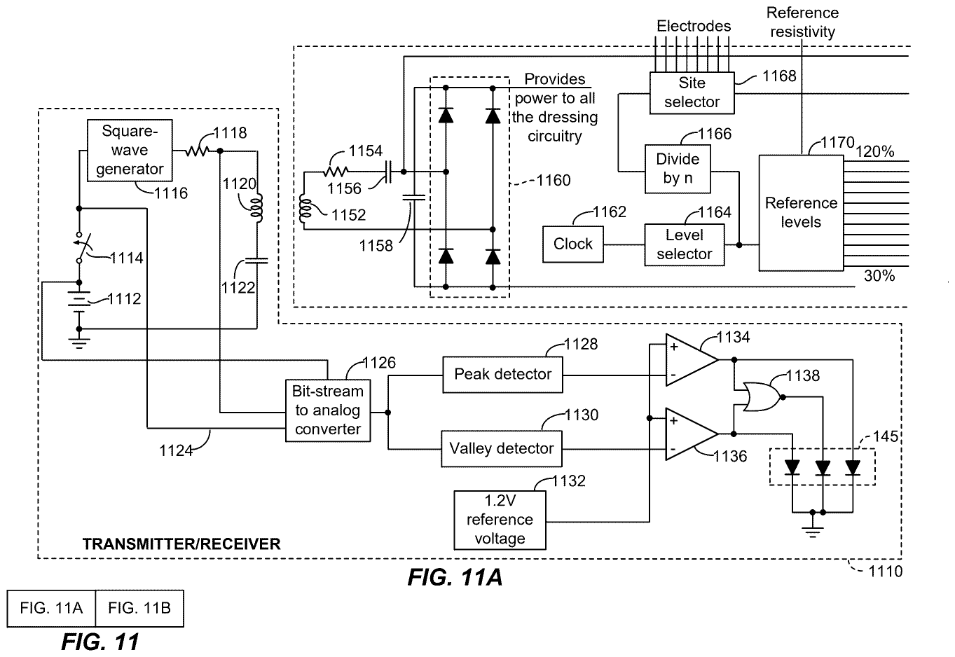

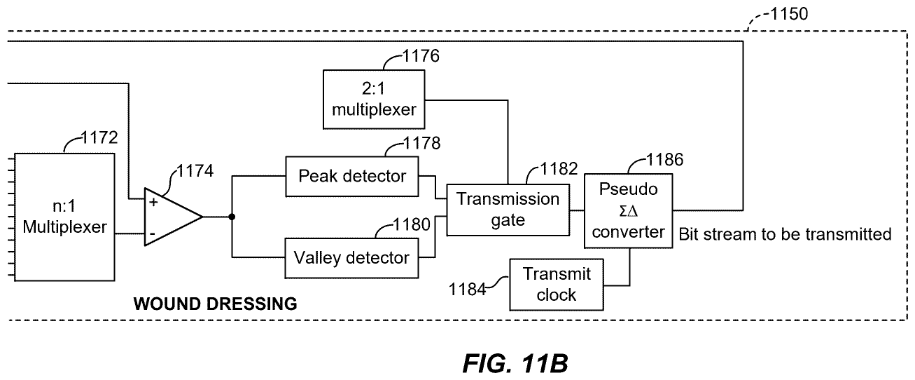

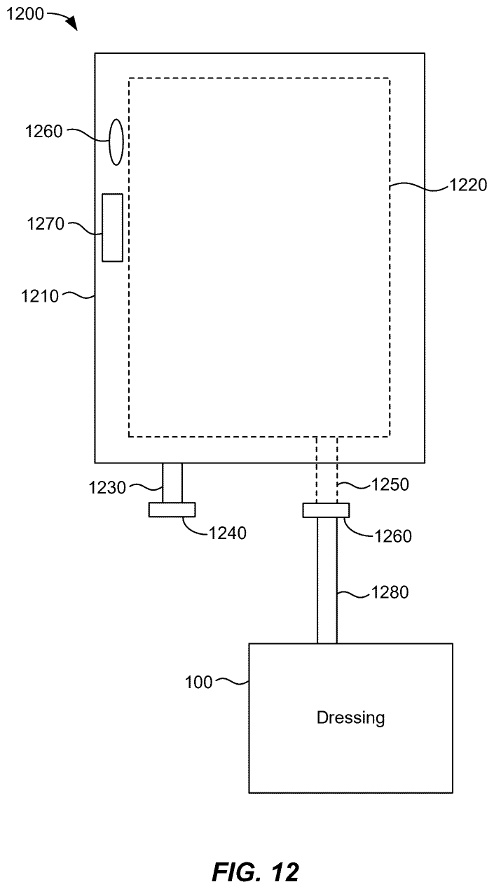

FIG. 11, illustrated in full by FIGS. 11A and 11B, is a simplified schematic diagram illustrating a wound dressing system including a transmitter/receiver and wound dressing according to an embodiment of the present invention. As illustrated in FIG. 11, transmitter/receiver 1110 is paired with wound dressing 1150 in order to transmit power and data between transmitter/receiver 1110 and wound dressing 1150.

Transmitter/receiver 1110 includes power supply 1112, for example a battery, and switch 1114. Square wave generator 1116 generates a square wave that passes through resistor 1118, inductor 1120, and capacitor 1122. The voltage between resistor 1118 and inductor 1120 is provided to bitstream to analog converter 1126 along with the voltage provided by power supply 1112 when switch 1114 is closed using lead 1124. When the square wave generator 1116 is turned off by the opening of switch 1114, that event is detected by circuitry in bit-stream to analog converter 1126, which then detects the time-varying voltage at the junction of resistor 1118 and inductor 1120. The output of bitstream to analog converter 1126 is connected to peak detector 1128 and valley detector 1130. In conjunction with a reference voltage 1132, for example, a 1.2 V reference voltage, comparator 1134 and comparator 1136 compare the output of peak detector 1128 and valley detector 1130, respectively to reference voltage 1132 to produce outputs that is provided to XNOR gate 1138. Accordingly, transmitter/receiver 1110 is able to produce output that is displayed to a user using output indicator 145.

Wound dressing 1150 is inductively coupled to transmitter/receiver 1110 using inductor 1152, which is connected to resistor 1154 and capacitor 1156. Using capacitor 1156 and capacitor 1158, circuit block 1160 is able to provide power to all of the wound dressing circuitry. During operation, clock 1162 provides an input to level selector 1164, which can select one of the reference levels 1170 previously discussed in relation to FIG. 3. Divide by N circuit 1166 is connected to level selector 1164 and site selector 1168 in order to determine which electrode(s) are sampled during the measurement process. Thus, as discussed in relation to FIGS. 3 and 4, both multi-reference and multi-site implementations are included within embodiments of the present invention.

The reference levels (e.g., the 30% to 120% resistance values illustrated in FIG. 11A) are connected to n:1 multiplexer 1172 illustrated in FIG. 11B. The output of site selector 1168 and the reference levels are provided to voltage comparator 1174, which is connected to peak detector 1178 and valley detector 1180. Driven by 2:1 multiplexer 1176, transmission gate 1182 receives the outputs of peak detector 1178 and valley detector 1180 as inputs and outputs a signal received by pseudo ΣΔ converter 1186, which, driven by transmit clock 1184, outputs the bit stream to be transmitted.

FIG. 12 is a simplified schematic diagram illustrating a nested container system according to an embodiment of the present invention. The nested container system illustrated in FIG. 12, also referred to as a composite bag or a bag-in-bag implementation, is suitable application of positive pressure, for example, carbon dioxide (CO₂) gas pressure to the iodinated wound dressing, for example, iodinated wound dressing illustrated in FIG. 6A.

Referring to FIG. 12, nested container system 1200 includes inner bag 1220 disposed inside outer bag 1210. The inner bag 1220 can be filled with normal saline solution, an iodine-containing solution, or the like, through tube 1250 sealed by port 1260. Port 1260 is also used to connect the inner bag 1220 to the dressing 100 through a tube 1280.

In some embodiments, the outer bag 1210 contains acetic acid 1260 or another mild acid introduced through tube 1230, sealed by port 1240. Outer bag 1210 also contains a vial 1270 of bicarbonate of soda or another mild base. Cracking the vial 1270 enables mixing of the acid and base, releasing CO₂ gas in the region between the exterior of inner bag 1220 and the interior of outer bag 1210. Since outer bag 1210 is not substantially expandable, the CO₂ gas formed in the space between the interior of outer bag 1210 and the exterior of inner bag 1220 compresses inner bag 1220, forcing the contents present in inner bag 1220 through tube 1250, port 1260, tube 1280, and into dressing 100.

In other embodiments, port 1240 includes an optional bladed structure 1242 that can be used to pierce the seal on a CO₂ cartridge (not shown) when the seal of the CO₂ cartridge is pressed into contact with the optional bladed structure 1242. In these embodiments, CO₂ gas can flow through the optional bladed structure into the space between the interior of outer bag 1210 and the exterior of inner bag 1220 in order to compresses inner bag 1220 and force the contents present in inner bag 1220 through tube 1250, port 1260, tube 1280, and into dressing 100 as discussed in the embodiment utilizing an acid/base reaction to generate the gas pressure. Thus, embodiments of the present invention enable either gas pressure based on mixing of fluids/solids present in the nested container system at manufacturing or provision of gas pressure from an external gas source, for example, a CO₂ cartridge.

In some embodiments, port 1240 can be replaced by a single opening with a female Leuer lock so that a male Leuer lock can be screwed into the top of the female Leuer lock in order to introduce gas pressure in the space between the inner bag and the outer bag. Similarly, port 1260 can be replaced with a similar system in order to provide for or reloading the wound dressing with iodine.

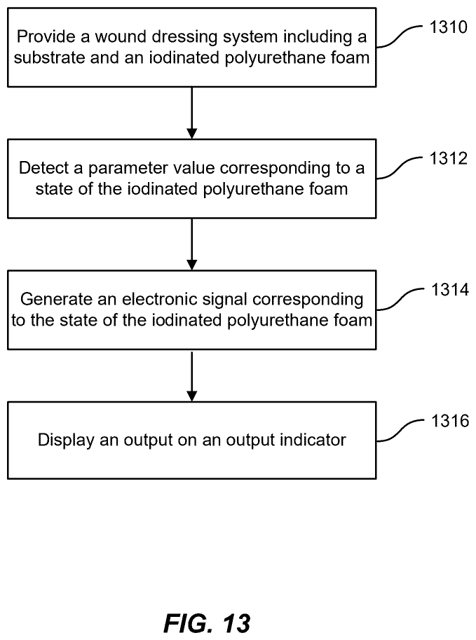

FIG. 13 is a simplified flowchart illustrating a method of operating a wound dressing system according to an embodiment of the present invention. As illustrated in FIG. 13, the method 1300 includes providing a wound dressing including a substrate and an iodinated polyurethane foam (1310) and detecting, using one or more sensors coupled to the substrate, a parameter value corresponding to a state of the iodinated polyurethane foam (1312). The substrate can be flexible. Also, the substrate can include a wound-contact layer, a sensor layer, a signal-routing layer, and a protective outer film. The one or more sensors can be implemented as a sensor array including at least two resistors, a first resistor of the at least two resistors being positioned at a central region of the wound dressing and a second resistor of the at least two resistors being positioned at a peripheral region of the wound dressing. Moreover, the one or more sensors can be implemented as a sensor array including at least two temperature sensors, a first temperature sensor of the at least two temperature sensors being positioned at a central region of the wound dressing and a second temperature sensor of the at least two temperature sensors being positioned at a peripheral region of the wound dressing.

The one or more sensors can include a resistor in order to measure a parameter value of resistance corresponding to a state of fluid level in the iodinated polyurethane foam. As described herein, as the fluid level decreases, the resistance of the iodinated polyurethane foam increases, indicating that the iodine solution needs to be replenished. Thus, based on the measured parameter value, the method can further include flowing an iodine solution into the iodinated polyurethane foam to increase a fluid level in the iodinated polyurethane foam. The one or more sensors can include a thermistor in order to measure a parameter value of temperature corresponding to a state of infection in the iodinated polyurethane foam. As described herein, if the wound is becoming infected or more infected, the temperature of the iodinated polyurethane foam increases, indicating that the iodine solution or the wound dressing needs to be replenished, antibiotics need to be utilized, or the like. The one or more sensors can include ion-sensitive field effect transistor in order to measure a parameter value of pH level corresponding to a state of liquid level or a state of infection in the iodinated polyurethane foam. As described herein, if CO₂ is lost from the wound surface, the acid pH of the wound may decrease, adversely impacting wound healing.

The method also includes generating an electronic signal, using electronics operatively connected to the one or more sensors, corresponding to the state of the iodinated polyurethane foam (1314) and displaying an output on an output indicator coupled to the electronics (1316). In one implementation, the electronics can include a resistance comparison circuit and the method can include comparing a resistivity measurement to a reference resistor and determining an iodine concentration in the wound dressing based on the comparison. The output indicator can be physically separated from the wound dressing. In some embodiments, the method includes inductively powering the one or more sensors and the electronics, whereas in other embodiments, a battery is coupled to the substrate in order to power the one or more sensors and the electronics. The output indicator can be implemented as an array of LEDs and displaying the output on the output indicator can include lighting one or more LEDs to indicate wound status by color code as described more fully herein.

It should be appreciated that the specific steps illustrated in FIG. 13 provide a particular method of operating a wound dressing according to an embodiment of the present invention. Other sequences of steps may also be performed according to alternative embodiments. For example, alternative embodiments of the present invention may perform the steps outlined above in a different order. Moreover, the individual steps illustrated in FIG. 13 may include multiple sub-steps that may be performed in various sequences as appropriate to the individual step. Furthermore, additional steps may be added or removed depending on the particular applications. One of ordinary skill in the art would recognize many variations, modifications, and alternatives.

### Artificial Intelligence (AI) Application

AI makes the sustained and smart release of aqueous iodine from the wound dressing described herein possible in order to kill bacteria in open wounds and burns. As discussed in relation to FIG. 8A, the liquid port 850 present on the back of the wound dressing (i.e., the side of the wound dressing facing away from the skin) will allow the attachment of an aqueous iodine solution reservoir (not shown). The electronic iodine sensors at the surface of the wound can measure the concentration in parts per million of iodine being released into the wound. If the measured concentration reaches 2 ppm or less, the AI chip in the application software will compress the reservoir to release iodine until the concentration reaches a preset amount. This preset amount can range from 2 ppm to 12 ppm or concentrations in this range. This ensures continuity of the concentration to destroy bacteria while being non-injurious to tissue. It is known that the concentration of iodine in the sponge acts by Fick's law to travel to a lesser concentration, which is known to elute between 2 ppm and 10 ppm.

The mechanisms utilized to cause the release of aqueous iodine into the wound dressing are electromechanical and will generate a pressure gradient as well as the concentration gradient.

Some embodiments of the wound dressing may rely on chemical gradients. As discussed in relation to FIG. 8A, the substrate 812 of the wound dressing 800 can be fabricated using an optically clear film that will allow for the visualization of the orange-yellow appearance of the iodinated foam 810. The substrate 812, which can be considered a clear portion of the wound dressing, will also allow the optical determination of the concentration of the iodine by spectrophotometry. This will be possible by establishing accurate iodine concentrations that are reproducible during the manufacture. As iodine is lost to the foam, spectrophotometric concentrations will change and will be utilized to check the accuracy of the electronic determination of the iodine concentration.

Moreover, the iodine utilized to create the iodinated foam will serve to sterilize the dressing by providing a concentration that kills all bacteria incorporated in 24 hours or less.

As described herein, the iodinated, intelligent wound dressing systems provided by embodiments of the present invention can utilize AI to improve, for example, optimize outcomes. Moreover, the wound dressing systems described herein can be adapted to different environments, various medications, patient interface (dressing) materials, wound configurations, and available equipment and patient conditions.

Various examples of the present disclosure are provided below. As used below, any reference to a series of examples is to be understood as a reference to each of those examples disjunctively (e.g., “Examples 1-4” is to be understood as “Examples 1, 2, 3, or 4”).

Example 1 is a wound dressing system comprising: a wound dressing including a substrate and an iodinated polyurethane foam; one or more sensors coupled to the substrate; electronics operatively connected to the one or more sensors; a power source; and an output indicator coupled to the electronics.

Example 2 is the wound dressing system of example 1 wherein the one or more sensors comprise a resistor.

Example 3 is the wound dressing system of example(s) 1-2 wherein the one or more sensors comprise a thermistor.

Example 4 is the wound dressing system of example(s) 1-3 wherein the one or more sensors comprise an ion-sensitive field effect transistor.

Example 5 is the wound dressing system of example(s) 1-4 wherein the substrate is flexible.

Example 6 is the wound dressing system of example(s) 1-5 wherein the one or more sensors are configured to contact a fluid present in the iodinated polyurethane foam.

Example 7 is the wound dressing system of example(s) 1-6 wherein the power source comprises a battery.

Example 8 is the wound dressing system of example(s) 1-7 further comprising a therapeutic delivery module operatively connected to the electronics and in fluid communication with the iodinated polyurethane foam.

Example 9 is the wound dressing system of example(s) 1-8 further comprising a transmitter coupled to the substrate, wherein the output indicator is physically separated from the wound dressing.

Example 10 is the wound dressing system of example(s) 1-9 wherein the substrate comprises a wound-contact layer, a sensor layer, a signal-routing layer, and a protective outer film.

Example 11 is the wound dressing system of example(s) 1-10 wherein the one or more sensors comprises a sensor array including at least two resistors, a first resistor of the at least two resistors being positioned at a central region of the wound dressing and a second resistor of the at least two resistors being positioned at a peripheral region of the wound dressing.

Example 12 is the wound dressing system of example(s) 1-11 wherein the one or more sensors comprises a sensor array including at least two temperature sensors, a first temperature sensor of the at least two temperature sensors being positioned at a central region of the wound dressing and a second temperature sensor of the at least two temperature sensors being positioned at a peripheral region of the wound dressing.

Example 13 is the wound dressing system of example(s) 1-12 wherein the electronics include a resistance comparison circuit configured to determine iodine concentration in the wound dressing by comparing a resistivity measurement to a reference resistor.

Example 14 is the wound dressing system of example(s) 1-13 wherein the power source comprises an inductive power source.

Example 15 is the wound dressing system of example(s) 1-14 further comprising edge-seal integrity monitoring sensors positioned at a peripheral region of the wound dressing.

Example 16 is the wound dressing system of example(s) 1-15 wherein the output indicator comprises at least one light emitting diode (LED) configured to indicate wound status by color code.

Example 17 is a method of operating a wound dressing system, the method comprising: providing a wound dressing including a substrate and an iodinated polyurethane foam; detecting, using one or more sensors coupled to the substrate, a parameter value corresponding to a state of the iodinated polyurethane foam; generating an electronic signal, using electronics operatively connected to the one or more sensors, corresponding to the state of the iodinated polyurethane foam; displaying an output on an output indicator coupled to the electronics.

Example 18 is the method of example 17 wherein the one or more sensors comprise a resistor.

Example 19 is the method of example(s) 17-18 wherein the one or more sensors comprise a thermistor.

Example 20 is the method of example(s) 17-19 wherein the one or more sensors comprise an ion-sensitive field effect transistor.

Example 21 is the method of example(s) 17-20 wherein the substrate is flexible.

Example 22 is the method of example(s) 17-21 wherein the state of the iodinated polyurethane foam comprises a fluid level.

Example 23 is the method of example(s) 17-22 further comprising flowing an iodine solution into the iodinated polyurethane foam to increase a fluid level in the iodinated polyurethane foam.

Example 24 is the method of example(s) 17-23 wherein the output indicator is physically separated from the wound dressing.

Example 25 is the method of example(s) 17-24 wherein the substrate comprises a wound-contact layer, a sensor layer, a signal-routing layer, and a protective outer film.

Example 26 is the method of example(s) 17-25 wherein the one or more sensors comprises a sensor array including at least two resistors, a first resistor of the at least two resistors being positioned at a central region of the wound dressing and a second resistor of the at least two resistors being positioned at a peripheral region of the wound dressing.

Example 27 is the method of example(s) 17-26 wherein the one or more sensors comprises a sensor array including at least two temperature sensors, a first temperature sensor of the at least two temperature sensors being positioned at a central region of the wound dressing and a second temperature sensor of the at least two temperature sensors being positioned at a peripheral region of the wound dressing.

Example 28 is the method of example(s) 17-27 wherein: the electronics include a resistance comparison circuit; and the method further comprises: comparing a resistivity measurement to a reference resistor; and determining an iodine concentration in the wound dressing based on the comparison.

Example 29 is the method of example(s) 17-28 further comprising inductively powering the one or more sensors and the electronics.

Example 30 is the method of example(s) 17-29 wherein: the output indicator comprises an array of light emitting diodes (LEDs); and displaying the output on the output indicator comprises lighting one or more LEDs to indicate wound status by color code.

Various features described herein, e.g., methods, apparatus, computer readable media and the like, can be realized using a combination of dedicated components, programmable processors, and/or other programmable devices. Some processes described herein can be implemented on the same processor or different processors. Where some components are described as being configured to perform certain operations, such configuration can be accomplished, e.g., by designing electronic circuits to perform the operation, by programming programmable electronic circuits (such as microprocessors) to perform the operation, or a combination thereof. Further, while the embodiments described above may make reference to specific hardware and software components, those skilled in the art will appreciate that different combinations of hardware and/or software components may also be used and that particular operations described as being implemented in hardware might be implemented in software or vice versa.

Detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology is used in the description for convenience of reference only and is not limiting. For example, up, down, front, back, right and left refer to the embodiments as orientated in the view being referred to. The words, “inwardly” and “outwardly” refer to directions toward and away from, respectively, the geometric center of the aspect being described and designated parts thereof. This terminology will include the words specifically mentioned, derivatives thereof and words of similar meaning.

Details are given in the above description to provide an understanding of the embodiments. However, it is understood that the embodiments may be practiced without some of the specific details. In some instances, well-known circuits, processes, algorithms, structures, and techniques are not shown in the figures.

While the principles of the disclosure have been described above in connection with specific apparatus and methods, it is to be understood that this description is made only by way of example and not as limitation on the scope of the disclosure. Embodiments were chosen and described in order to explain principles and practical applications to enable others skilled in the art to utilize the invention in various embodiments and with various modifications, as are suited to a particular use contemplated. It will be appreciated that the description is intended to cover modifications and equivalents.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc.

A recitation of “a”, “an”, or “the” is intended to mean “one or more” unless specifically indicated to the contrary.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of embodiments of the invention has been presented for the purposes of illustration and description. It is not intended to be exhaustive or to limit the invention to the precise form described, and many modifications and variations are possible in light of the teaching above. The embodiments were chosen and described in order to explain the principles of the invention and its practical applications to thereby enable others skilled in the art to utilize the invention in various embodiments and with various modifications as are suited to the particular use contemplated.

What is claimed is:

1. A wound dressing system comprising:
a wound dressing including a substrate and an iodinated polyurethane foam;
one or more sensors coupled to the substrate;
electronics operatively connected to the one or more sensors;
a power source; and
an output indicator coupled to the electronics.

2. The wound dressing system of claim 1 wherein the one or more sensors comprise a resistor.

3. The wound dressing system of claim 1 wherein the one or more sensors comprise a thermistor.

4. The wound dressing system of claim 1 wherein the one or more sensors comprise an ion-sensitive field effect transistor.

5. The wound dressing system of claim 1 wherein the substrate is flexible.

6. The wound dressing system of claim 1 wherein the one or more sensors are configured to contact a fluid present in the iodinated polyurethane foam.

7. The wound dressing system of claim 1 wherein the power source comprises a battery.

8. The wound dressing system of claim 1 further comprising a therapeutic delivery module operatively connected to the electronics and in fluid communication with the iodinated polyurethane foam.

9. The wound dressing system of claim 1 further comprising a transmitter coupled to the substrate, wherein the output indicator is physically separated from the wound dressing.

10. The wound dressing system of claim 1 wherein the substrate comprises a wound-contact layer, a sensor layer, a signal-routing layer, and a protective outer film.

11. The wound dressing system of claim 1 wherein the one or more sensors comprises a sensor array including at least two resistors, a first resistor of the at least two resistors being positioned at a central region of the wound dressing and a second resistor of the at least two resistors being positioned at a peripheral region of the wound dressing.

12. The wound dressing system of claim 1 wherein the one or more sensors comprises a sensor array including at least two temperature sensors, a first temperature sensor of the at least two temperature sensors being positioned at a central region of the wound dressing and a second temperature sensor of the at least two temperature sensors being positioned at a peripheral region of the wound dressing.

13. The wound dressing system of claim 1 wherein the electronics include a resistance comparison circuit configured to determine iodine concentration in the wound dressing by comparing a resistivity measurement to a reference resistor.

14. The wound dressing system of claim 1 wherein the power source comprises an inductive power source.

15. The wound dressing system of claim 1 further comprising edge-seal integrity monitoring sensors positioned at a peripheral region of the wound dressing.

16. The wound dressing system of claim 1 wherein the output indicator comprises at least one light emitting diode (LED) configured to indicate wound status by color code.

17. A method of operating a wound dressing system, the method comprising:
providing a wound dressing including a substrate and an iodinated polyurethane foam;
detecting, using one or more sensors coupled to the substrate, a parameter value corresponding to a state of the iodinated polyurethane foam;
generating an electronic signal, using electronics operatively connected to the one or more sensors, corresponding to the state of the iodinated polyurethane foam;
displaying an output on an output indicator coupled to the electronics.

18. The method of claim 17 wherein the state of the iodinated polyurethane foam comprises a fluid level, the method further comprising flowing an iodine solution into the iodinated polyurethane foam to increase a fluid level in the iodinated polyurethane foam.

19. The method of claim 17 wherein:
the electronics include a resistance comparison circuit; and
the method further comprises:
comparing a resistivity measurement to a reference resistor; and
determining an iodine concentration in the wound dressing based on the comparison.

20. The method of claim 17 further comprising inductively powering the one or more sensors and the electronics.
